(19) United States
(12) Patent Application Publication
(10) Pub. No.: US 2024/0079095 A1
(43) Pub. Date: Mar. 7, 2024

(54) Using supplementary and secondary alignments to improve compression of genomic alignment files

(71) Applicant: Divon Mordechai Lan, Bangkok (TH)

(72) Inventor: Divon Mordechai Lan, Bangkok (TH)

(21) Appl. No.: 17/938,980

(22) Filed: Sep. 7, 2022

(51) Int. Cl. G16B 50/50; G06F 16/174; G16B 30/10

(52) U.S. Cl. CPC G16B 50/50; G06F 16/1744; G16B 30/10

(57) ABSTRACT

The present invention discloses an advance methodology for using supplementary and secondary alignments to improve compression of genomic alignment file and method for exploiting information redundancies that exist in a between a primary alignment and secondary or supplementary alignments of the same read, to improve the compression of aligned genomic data. The method is implemented to compress SAM/BAM files, but in the future, it could be used to compress other file formats of aligned genomic data.

### DESCRIPTION OF THE RELATED ART

Genomics is a field of active research today. An understanding of the genome variation may enable researchers to fully understand the issues of genetic susceptibility and pharmacogenomics of drug response for all individuals as well as personalized molecular diagnostic tests. For such research or medical purposes, genetic material obtained directly from either a biological or an environmental sample is generally sequenced into a plurality of sequences, called “reads”. A facility, such as a research laboratory or a clinic involved in genomic study typically uses high capacity platforms, such as next generation sequencing (NOS) platforms capable of sequencing a large number of samples per year. The reads generated may be further processed by aligning them to a set of reference sequences, such as a reference genome. Generally, the aligned reads, may be stored for future studies for further analysis. Thus, each year, genomic data, including files containing aligned reads, are generated in huge volumes, in the range of petabytes (PB), and stored in the repositories.

RNA and DNA are essentially chains of 4 possible bases (A,C,G,T in the case of DNA and A,C,G,U in the case of RNA). These chains can be as short as a few hundred bases as in the case of some RNA molecules or as long as hundreds of millions of bases as in the case of some DNA chromosomes. Scientists and clinicians are often interested in knowing the exact sequence of bases in a particular DNA or RNA molecule. To achieve that, they “sequence” the DNA or RNA molecule in a sequencing machine. However, sequencing machines can only handle relatively short chains of bases (in current technologies, this is between tens of bases to millions of bases depending on the sequencing technology), so the molecule needs to be first chopped up into a large number of fragments for sequencing. The output of the sequencing process is a computer file which contains “reads”. Each read contains the sequence of bases for one of the fragments. A read looks something like this (the format shown here is called FASTQ, that is used by many, but not all, sequencing technologies):
@QNAME1
X₀X₁X₂X₃X₄X₅X₆X₇X₈X₉
+
Q₀Q₁Q₂Q₃Q₄Q₅Q₆Q₇Q₈Q₉

The first line is the read name and some other metadata. The second line is the actual DNA/RNA sequence of the fragment—each Xi in this example appears in actual FASTQ files as one of the four bases (nucleotides) A,C,G,T or a code representing a combination of possible nucleotides, such as N. The fourth line are “base qualities”—each Qi appears in actual FASTQ file as an ASCII character of value between 33 and 126, corresponding to the respective base and represents the degree in which the system is confident that the base in the molecule is actually the bass listed. The file with the reads can contain thousands to billions of such reads. The read in this example has 10 bases, but in actual FASTQ files reads may have any number of bases.

For many species (including humans), there exists at least one “reference genome” which is a set of published sequences describing the genome of one or more individuals of that species. The next step in an analysis, once we have the reads file, is often mapping (also known as aligning) the reads generated by sequencer to a set of sequences, which is typically, but not necessarily, a reference genome (for example, It might also be a transcriptome, or a set of sequences that contains both the genome sequences as well as other sequences). Of course, every individual is different and hence has a genome that is slightly different than the reference genome, so the reads might not map perfectly to the reference sequences. They also might not map perfectly because of errors in the sequencing process, or because the read is actually not from the intended molecule but from contamination, or because the DNA or RNA is mutated (for example, it is from a cancer cell), or for other reasons. Sometimes, reads might map well to multiple locations in the reference sequences so we have uncertainty of where they actually cam from. The software that maps the read data to a reference genome is called an aligner. Many aligner software packages exist, each with their own advantages and disadvantages. The output of an aligner is a file of aligned reads also called alignments. Most aligners output the alignments in either SAM or BAM file format (these are closely related formats), but some aligns use other file formats.

An alignment in the SAM format is a tab-separated textual line that looks something like this:
QNAME1 97 chr3 58492070 37 10M=58492070 51 X₀X₁X₂X₃X₄X₅X₆X₇X₈X₉ Q₀Q₁Q₂Q₃Q₄Q₅Q₆Q₇Q₈Q₉XT:A:U NM:i:0 SM:i:37 AM:i:37 X0:i:1 X1:i:0 XM:i:0 XO:i:0 XG:i0 MD:Z:51
   The first field is the read name (“QNAME1”), the 2nd field (“97”) in the FLAGS field, the 10th field (“X₀X₁X₂X₃X₄X₅X₆X₇X₈X₉”) represents a base (nucleotide) sequence of the read as explained in paragraph 0003 above, and the 11th (“Q₀Q₁Q₂Q₃Q₄Q₅Q₆Q₇Q₈Q₉”) is the base quality scores as explained in paragraph 0003 above. All the other fields describe the alignment—the position of the read in the reference sequences (fields 3,4) and other various metadata regarding properties of this read and its alignment to the genome. Similar to a FASTQ file, a SAM/BAM file often contains hundreds of millions of alignments.

Secondary alignments: sometimes the aligner finds several locations in the reference sequences that a given read can be mapped to, so it is unsure which is the true location. In this case, it will pick its best guess as primary alignment, but might also output some of the secondary alignments. In SAM format, them are two common ways in which aligners output secondary alignments:

Option 1: adding additional alignments to the file, and marking them as secondary by setting the “Secondary” bit (bit 8) in the FLAGS field. In Example 1, there are 3 alignments of the same read “QNAME1”. The 3rd one (with FLAG-16) is the primary alignment. The first two with bit 8 set in the FLAG (FLAG=256 or 272) an secondary alignments of the same read. One can notice there's redundancy between these 3 alignments—eg the sequence “X̅₉X̅₈X̅₇X̅₆X̅₅X̅₄X̅₃X̅₂X̅₁X̅₀” and the quality “Q₉Q₈Q₇Q₆Q₅Q₄Q₃Q₂Q₁Q₀” are the same in the 1st and 3rd alignment. Same of the other fields ae also related.

The 2nd alignment however, is reversed vs the 1st and 3rd alignments: this can be seen because bit 4 of its HAG is 0 (i.e. not reverse-complemented), while this bit is at for the 1st and 3rd alignments (i.e. they are reverse-complemented relative to the reference genome—indicated by the Xi and Qi series being in reverse order, and X̅ representing the complement base of X—A< >T and C< >G). Notice at the QUA, field of the second alignment “Q₀Q₁Q₂Q₃Q₄Q₅Q₆Q₇Q₈Q₉” is the reverse of the primary's QUAL field. The 2nd alignment's SEQ field “X₀X₁X₂X₃X₄X₅X₆X₇X₈X₉” is the “reverse-complement” of the primary's.

### EXAMPLE 1

QNAME1 272 chr9 11301 0 6M7198N4M*0 0 X̅₉X̅₈X̅₇X̅₆X̅₅X̅₄X̅₃X̅₂X̅₁X̅₀ Q₉Q₈Q₇Q₆Q₅Q₄Q₃Q₂Q₁Q₀ NH:i:6 HI:i3 AS:i:99 NM:i:0 MD:Z:101 XS:A:+
QNAME1 256 chr15 101978881 0 4M798N6M*0 0 X₀X₁X₂X₃X₄X₅X₆X₇X₈X₉ Q₀Q₁Q₂Q₃Q₄Q₅Q₆Q₇Q₈Q₉ NH:i:6 HI:i:6 AS:i:99 NM:i:0 MD:Z:101 XS:A:−
QNAME1 16 chr16 10869 0 2M798N8M*0 0 X̅₉X̅₈X̅₇X̅₆X̅₅X̅₄X̅₃X̅₂X̅₁X̅₀ Q₉Q₈Q₇Q₆Q₅Q₄Q₃Q₂Q₁Q₀ NH:i:6 HI:i:1 AS:i:99 NM:i:0 MD:Z:101 XS:A:+
   Option 2: listing secondary alignments in the alignment itself. There are multiple ways aligners do this, one of them is by using an XA:Z field:

### Example 2

QNAME1 163 chrM 621 60 10M=851 331 X₀X₁X₂X₃X₄X₅X₆X₇X₈X₉ Q₀Q₁Q₂Q₃Q₄Q₅Q₆Q₇Q₈Q₉ RG:Z:NA12878 XT:A:U NM:i:0 SM:i:23 AM:i:23 X0:i:1 X:i:1 XM:i:0 XOi0 XO:i:0 MD:Z:101 XA:Z:chr2,-149639318,10M,1;
In this case, the aligner has mapped the read named “QNAME1” to the chromosome chrM of the genome at position 62.1, but in the field XA:Z it also informs us that there is another possible alignment of this read—to chromosome chr2 at position 149639318. In other cases, the XA:Z could list multiple secondary alignments.

### Supplementary Alignments

Supplementary alignments: This happens in a situation where port of a read maps to one location in the reference sequences, but another part (or parts) of die read maps to other locations. In this case, one of the alignments would be the primary alignment, and supplementary alignments are described in the SA:Z field. Those supplementary alignments are also present in the file.

Example 3—the underlined fields in the first alignment contain information redundant with the undefined fields in the second alignment.
QNAME1 185 chr14 586158 0 2S8M=58615877 0 X̅₉X̅₈X̅₇X̅₆X̅₅X̅₄X̅₃X̅₂X̅₁X̅ Q₉Q₈Q₇Q₆Q₅Q₄Q₃Q₂Q₁Q₀ NM:i:0 MD:Z:19 AS:i19 XS:i:19 RG:Z:FD09254804 SA:Z:chr8,17711142,-5S4M1S,0,0: XA:Z:chr,-127358389,126S19M5S,0;
QNAME1 2233 chr8 17711142 0 5H4M1H=177111420 0 X̅₄X̅₃X̅₂X̅₁ Q₄Q₃Q₂Q₁ NM:i:0 MD:Z:19 AS:i:19 AS:i:0 RG:Z:FD09254804 SA:Z:chr14,58615877,-, 2S8M,0,0;
   Note that the supplementary alignment, in this case (but not always), contains only a subset of the sequence and base quality scores
   Note: some aligners use the SA:Z field described above to store secondary, rather than supplementary alignments. For the purposes of the current invention, we treat secondary and supplementary alignments the same.

There are multiple solutions that have been presented in prior at for compression SAM/BAM files. It has to be noted that the current invention proposes an advancement in providing a method which relates to better exploiting information redundancies that exist in a between a primary alignment and secondary or supplementary alignments of the same read, as well as redundancies between a plurality of secondary or supplementary alignments of the same read, to improve the compression of aligned genomic data. In Genozip the invention is implemented to compress SAM/BAM files, but in the future it could be used to compress other file formats of aligned genomic data.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” ae intended to include the plural forms as well as the singular forms, unless the context clearly indicates otherwise. It will be further understood that the terms “comprises” and/or “comprising,” when used in this specification, specify the presence of stated features, steps, operations, elements, and/or component, but do not preclude the presence or addition of one or more other features, steps, operations, elements, components, and/or groups thereof.

The present invention is directed to systems and methods for compression of genomic data files are described herein. Generally, genetic material extracted directly from either a biological or an environmental sample is processed and stored as genomic data for research or medical purposes. The genomic data typically includes the genetic material sequenced into a plurality of sequences, called reads, which may then be aligned to a set of reference sequences, including (but not limited to) a reference genome. An aligned read, as will be known to a person skilled in the at, typically includes data describing the nucleotide sequence of this read, the base quality scores, the location in the reference sequences to which this read was aligned, as well a other information about the read and/or its alignment.

In order to increase the efficiency of the repositories in storing the genomic data and relates to exploiting information redundancies that exist in a between a primary alignment and secondary or supplementary alignments of the same read, and/or between a plurality of secondary or supplementary alignments of the same read to improve the compression of aligned genomic data. In Genozip the invention is implemented to compress SAM/BAM files, but in the future it could be used to compress other file formats of aligned genomic data.

The method defines the group of alignments which includes the primary alignment and the related secondary and/or supplementary alignments as a“sag”. In case of paired-end reads, each read in the pair would be in separate sag.

Part 1: Exploiting Redundancies Between Alignments of the Same Sag:

SEQ (sequence): we express the nucleotide sequence (SEQ) of one alignment in the sag in relation to that of another alignment in the same sag: for the overlapping parts of SEQ, we store just a description of the overlap (i.e. the information allowing copying of the correct part of the sequence from the related alignment) rather than store the SEQ itself. If the two alignments have a different orientation (i.e. different Rev-Comp bit value of the FLAG field), the comparison is done after reverse complementing one of them.

base quality cares (QUAL): we express QUAL of one alignment in the sag in relation to that of another alignment in the same sag: for overlapping parts of QUAL, we store just a reference to the other alignment rather than store the entire QUAL itself. In some cases, the QUAL of the reads in the sag might differ due to further processing that occurred on the quality data (for example, a procedure known as base quality recalibration). In this case, we also store the base quality scores that are different between each respective quality score of the two reads, or, in another embodiment, the base-wise difference in the base quality score between the two alignments. These differences, since they tend to be 0 or near 0 for most bases of the reads, usually compress significantly better than compressing the base quality score data itself. If the two alignments have a different orientation (i.e. different Rev-Comp bit value of the FLAG field), the comparison is done after reversing one of them.

Additional fields: In several other fields we may also benefit from exploiting redundancies between alignments in the same sag: For example the SA:Z field of an alignment in a sag might be predicted from the fields of the fields SA:Z, RNAME, POS, FLAG, CIGAR and NM:i of another alignment in the same sag. In the common case where the prediction is correct, we can store a single bit indicating that the prediction is correct instead of storing the SA:Z field. Similarly, the values of the RNAME, POS, NM:i and CIGAR fields, as well as the Rev-Comp bit in FLAG, can be predicted from the SA:Z field of another read in the same sag. Likewise, the fields NH:i can be predicted by NH:i of another alignment in the same sag, CP:i can be predicted by the POS field of one of the alignments of the same sag.

When compressing a SAM or BAM file, Genozip divides the file into blocks of alignments, called vblocks, and compresses each vblock separately, using a compute thread. If two reads in the same sag are close to each other in the file such that they appear in the same vblock, the compute thread responsible for compressing the vblock can exploit these redundancies directly as explained above. However, in many cases the alignments of a certain sag might be far away from each other in the file, such that they are present in different vblocks. In this case, Genozip removes the secondary and supplementary alignments from the vblock, as well as primary alignments which are known to have secondary or supplementary alignments in their sag (this might be due a presence of an SA:Z, or by NH:i field being 2 or greater, or by the presence of CC:Z/CP:i fields or by preprocessing the file ahead of compressing and directly finding sag), and creates separate vblocks containing only primary alignments, and other vblocks containing only secondary/supplementary alignments. When the primary alignments contained in primary-alignments only vblock are processed in their compute thread, exploitable information from these primary reads is also stored in RAM. The compute threads compressing the vblocks containing secondary/supplementary alignments am executed only after the compute threads compressing primary alignments have completed, and hence the exploitable information from the primary alignments is present in RAM. Then, when processing a secondary/supplementary alignment, the compute thread looks up its corresponding primary alignments in RAM, and if found, may exploit the data from the primary alignments to compress the secondary/supplementary alignments. When decompressing, a similar process is applied: the primary vblocks are read first, exploitable information is stored in RAM, and the secondary alignments, when decompressed, may refer to data from the primary alignments. Finally, a Genozip module called writer reassembles the alignments from the main, primary and secondary vblocks to their original order.

Part 2: Using Information Contained in the SA:Z and/or XA:Z Field to Improve Compression of the Sequence (SEQ Field).

In Genozip, when a compute thread compresses the SEQ field, using Example 3 above an example: “X̅9X̅8X̅7X̅6X̅5X̅4X̅3X̅2X̅1X̅0”

Rather than storing the entire SEQ data, it can use reference sequences (for example: the reference genome), the file name of which is provided by the user as a command line parameter, to improve compression: since the alignment tells us explicitly where the SEQ data appears in the reference sequence (sequence chr14 at position 58615877 in this Example 3) Genozip compares the given SEQ data to the indicated position in the indicated reference sequence, and stores a description of where and how SEQ is the same or differs from the reference sequence, instead of storing SEQ itself. Furthermore, even if the user does not provide the reference sequences, Genozip generates approximate reference sequences in memory while traversing the file, based on the alignments in the file itself.

Some of the bases in SEQ (namely those indicated by the CIGAR string to be soft-clips or insertions) might not appear in the reference sequence, in which case they need to be stored verbatim. The present invention improves on this point in the following way: if supplementary or secondary alignment ae indicated using an SA:Z or XA:Z field, then, for each base of SEQ that are soft clips or insertions according to CIGAR. Genozip uses the first alignment listed within SA:Z or XA:Z that does map that particular base to one of the reference sequences. Only if no alignments in SA:Z or XA:Z map the base against the genome, is the base stored verbatim.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims ae hereby incorporated into the Detailed Description, with each claim standing on its own as a separately claimed subject matter.

1: The innovative method of exploiting redundancies between primary and secondary/supplementary alignments of the same read or between two secondary/supplementary alignments of the same read in a genomic alignments file to better compress said genomic alignments file wherein;
As per claim 1, the genomic alignments files are in the SAM or BAM format;
As per claim 1, the compressing SEQ data of an alignment that has a forward or reverse-complement overlap with the other alignment, by storing only a description of the overlap instead of the SEQ data itself;
As per claim 1, the compressing QUAL data of an alignment has a forward or reverse overlap with the other alignment, by storing only a description of the overlap instead of the QUAL data itself;
As per claim 1, the compressing QUAL data of an alignment that has a forward or reverse overlap with the other alignment, by storing only a description of the overlap and the base quality stores within the overlap that are different from the other alignment; and,
As per claim 1, the compressing a field of an alignment can be predicted from the other alignment, possibly in combination with additional parameters, by storing the prediction and parameters rather than the field itself.
As per claim 1, the two alignments can be located anywhere in the genomic alignments file, without restriction on the distance within the file between the two alignments

2: The method of using information contained in an alignment, describing secondary or supplementary alignments of the same read, to improve the compression of the nucleotide sequence data of the alignment wherein:
As per claim 2, the file is in SAM or BAM format, and the secondary or supplementary alignments are described in the SA:Z and/or XA:Z fields.
